(19) United States
(12) Patent Application Publication
(10) Pub. No.: US 2019/0218479 A1
(43) Pub. Date: Jul. 18, 2019

(54) Stabilized Liquid Peroxide Compositions

(71) Applicants: Novozymes A/S, Bagsvaerd (DK); Arkema Inc., King of Prussia, PA (US)

(72) Inventors: Victor Casella, Raleigh, NC (US); Lise Munch Mikkelsen, Roedovre (DK); Priscilla Haasis, Louisburg, NC (US); Danielle Rhine, Roanoke, VA (US)

(73) Assignees: Novozymes A/S, Bagsvaerd (DK); Arkema Inc., King of Prussia, PA (US)

(21) Appl. No.: 16/304,912

(22) PCT Filed: May 30, 2017
(86) PCT No.: PCT/US2017/034995

§ 371 Date: Nov. 27, 2018

Related U.S. Application Data
(60) Provisional application No. 62/343,213, filed on May 31, 2016.

(51) Int. Cl. C11D 3/386; C11D 3/39

(52) U.S. Cl. CPC C11D 3/38663; C12N 9/6424; C11D 3/38618; C11D 3/3947

(57) ABSTRACT

The invention provides a liquid peroxide composition comprising a subtilisin having a M222 substitution (BPN′ numbering) and a peptide based subtilisin stabilizer.

### REFERENCE TO A SEQUENCE LISTING

This application contains a Sequence Listing in computer readable form. The computer readable form is incorporated herein by reference.

### FIELD OF THE INVENTION

The present invention relates to stabilization of proteases in liquid compositions comprising peroxide.

### BACKGROUND

Various strategies have been used to render bleach and enzymes compatible in detergent during storage, and in use.

DE102014008586 incorporates bleaches or bleach components into a wax matrix, which is then coated with an ionic polymer, forming particles which can be added to liquid detergents containing enzymes. This strategy supposedly allows the enzymes to work before the bleach is released.

WO 2015/095439 discloses stable, aqueous, liquid compositions comprising both an enzyme and a peroxide or peroxide source. The compositions contain a compatibilizer package having at least one compound chosen from enzyme stabilizers and peroxide stabilizers.

The present invention provides a different strategy, which involves use of a specific group of peptide aldehyde protease stabilizers, or derivatives thereof, that allows liquid co-formulation of proteases and peroxide containing bleach.

WO 98/13458, WO 94/04651, WO 98/13460, WO 95/25791, and WO 2009/118375 disclose liquid detergents with a subtilisin-type protease stabilized by a peptide aldehyde. WO 2011/036153 discloses that the addition of a peptide aldehyde to a particulate subtilisin-containing detergent can improve the detergency.

It is well known that aldehydes can form soluble adducts with NaHSO₃ (bisulfite or hydrosulfite adducts) and that peptide aldehydes tend to be sparingly water soluble. Peptide aldehyde hydrosulfite adducts, and their use in detergents, are disclosed in WO 2013/004636.

### SUMMARY

The present invention provides a liquid composition comprising the following key components:

(a) a peroxide or peroxide source,
(b) a subtilisin having an amino acid sequence comprising a substitution in a position corresponding to M216 of SEQ ID NO: 1,
(c) a peptide aldehyde or ketone subtilisin stabilizer, or a hydrosulfite adduct thereof, and
(d) optionally a peroxide stabilizer.

In an embodiment, the liquid composition is a cleaning or detergent composition comprising a surfactant (such as a nonionic and/or anionic surfactant) and other ingredients used in detergent and cleaning compositions.

Other aspects and embodiments of the invention are apparent from the description and examples. Unless otherwise indicated, all percentages indicated in the description, examples and claims are by weight.

### DETAILED DESCRIPTION

The advantage of the present invention is that liquid cleaning compositions containing peroxide (such as hydrogen peroxide), a subtilisin enzyme, and a subtilisin stabilizer are now commercially feasible (i.e., can be delivered without resorting to special multichamber packaging or encapsulation technology). The combined advantage of peroxide and enzyme technology provides more robust overall cleaning and stain removal performance than either technology alone. Peroxides are effective on “bleachable stains”, for example, stains containing chromophores (e.g., wine, coffee, tea, etc.), and enzymes are effective removing complex stains that contain, for example, proteins, starches, complex carbohydrates (e.g., guar, locust bean, etc.), and/or triglycerides that are not effectively removed by peroxide alone at typical peroxide use levels in laundry or dish wash applications. Examples of potential applications (but not limiting) include liquid and gel laundry (pretreaters and regular detergents); auto- and manual dishwash gels and liquids; liquid based surgical cleaners (pre-disinfection step).

### Conventions for Designation of Subtilisin Variants

For purposes of the present invention, the subtilisin amino acid sequence disclosed in SEQ ID NO: 1 is used to determine the corresponding amino acid residue in another subtilisin. The amino acid sequence of another subtilisin is aligned with the amino acid sequence disclosed in SEQ ID NO: 1, and based on the alignment, the amino acid position number corresponding to any amino acid residue in the subtilisin amino acid sequence disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another subtilisin can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other subtilisin has diverged from the amino acid sequence of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

### Substitutions

For an amino acid substitution, the following nomenclature is used:

Original amino acid, position, substituted amino acid.

Accordingly, the substitution of methionine at position 216 with serine is designated as “Met216Ser” or “M216S”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

### Subtilisin

The subtilisin enzymes for use in the present invention include those of bacterial, fungal, plant, viral or animal origin, e.g., vegetable or microbial origin. Microbial origin is preferred. Chemically modified or protein engineered mutants are included.

The subtilisin has an amino acid sequence, which comprises a substitution of the amino acid corresponding to amino acid position M216 of SEQ ID NO: 1, when the amino acid sequences are aligned as described above under ‘Conventions for designation of subtilisin variants’. Examples of such substitutions include, but are not limited to, M216A or M216S.

Position M216 of SEQ ID NO: 1 is equivalent to M222 using BPN′ numbering (and alignment).

In the context of the present invention, the subtilisin enzyme family (EC 3.4.21.62) shall be understood as described by Siezen et al., Protein Engng. 4 (1991) 719-737 and Siezen et al. Protein Science 6 (1997) 501-523. As described therein, the subtilisin family may be divided into 3 sub-groups, i.e. I-S1 (“true” subtilisins), I-S2 (highly alkaline proteases) and intracellular subtilisins.

Commercially available subtilisins, suitable for use in the present invention, include the subtilisins sold under the trade names Duralase™, Durazym™, Everlase™ (Novozymes A/S); and those sold under the trade names Maxapem™, Purafect Ox™, Purafect OxP™, Effectenz™ P1050, Effectenz™ P1060 (Genencor/Danisco/DuPont).

Other subtilisins, and variants thereof, can be used as a “starting point subtilisin” to produce a subtilisin for use in the present invention by making a M216 substitution (or M222 using BPN′ numbering), as described above, in the amino acid sequence of the “starting point subtilisin”.

Examples of such “starting point subtilisins” are those derived from Bacillus such as subtilisin lentus, Bacillus lentus, subtilisin Novo, subtilisin Carlsberg, Bacillus licheniformis, subtilisin BPN′, subtilisin 309, subtilisin 147 and subtilisin 168 described in WO 89/06279 and protease PD138 (WO 93/18140). Additional examples are described in WO 98/020115, WO 01/44452, WO 01/58275, WO 01/58276, WO 03/006602 and WO 04/099401.

Other examples of useful “starting point subtilisins” are the variants described in WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 43, 57, 61, 62, 68, 76, 87, 95, 96, 97, 98, 99, 100, 101, 102, 103, 104, 106, 118, 120, 123, 128, 129, 130, 156, 158, 160, 161, 167, 170, 182, 185, 188, 191, 194, 195, 199, 204, 205, 206, 209, 212, 217, 218, 224, 232, 235, 236, 245, 248, 252, 261, 262, 274 and 275, using the BPN′ numbering. The “starting point subtilisin” may comprise a substitution at one or more positions corresponding to positions 171, 173, 175, 179, or 180 of SEQ ID NO: 3 of WO 2004/067737.

More preferred the subtilisin variants may comprise one or more of the following substitutions: S3T, V41, S9R, S9E, A15T, K27R, *36D, N43R, G61E, G61D, N62D, N62E, V68A, N76D, N87S,R, *97E, A98S, S99G, S99D, S99A, S99AD, S101E, S101D, S101G, S101M, S101N, S101R, S101H, S103A, V104I, V104Y, V104N, S106A, G118V, G118R, H120D, H120N, N123S, S128L, P129Q, S130A, S156D, A158E, G160D, G160P, S161E, Y167A, R170S, Q182E, N185E, S188E, Q191N, A194P, G195E, V199M, N204D, V205I, Y209W, S212G, L217Q, L217D, N218D, N218S, A232V, K235L, Q236H, Q245R, N252K, N261W, N261D, N261E, L262E, L262D T274A, R275H (using BPN′ numbering).

Examples of commercially available proteases, which may be either combined with the subtilisin used in the present invention, or which may be used as a “starting point subtilisin” (as described above), include those sold under the trade names Alcalase™ Relase™ Savinase™ Primase™ Polarzyme™ Kannase™, Liquanase™, Ovozyme™, Coronase™, Blaze™, Neutrase™ and Esperase™ (Novozymes A/S); those sold under the tradename Maxatase™ Maxacal™, Puramax™, FN2™, FN3™, FN4™, Excellase™, Excellenz™ P1000, Excellenz™ P1250, Eraser™, Preferenz™ P100, Purafect Prime™, Preferenz™ P110, Effectenz™ P1000, Purafect™, Effectenz™ P2000, Purafast™, Properase™, Opticlean™ and Optimase™ (Genencor/Danisco/DuPont); Axapem™ (Gist-Brocases N.V.); BLAP (sequence shown in FIG. 29 of U.S. Pat. No. 5,352,604) and variants hereof (Henkel AG); and KAP (Bacillus alkalophilus subtilisin) from Kao.

In an embodiment, the subtilisin used in the invention is a variant of subtilisin 309 or subtilisin BPN′. Preferably, the amino acid sequence of the subtilisin has at least 60% sequence identity, preferably at least 70%, more preferably at least 80%, more preferably at least 90%, more preferably at least 95%, 96%, 97%, 98%, and most preferably 99% sequence identity to the amino acid sequence of SEQ ID NO: 1 or SEQ ID NO: 2. In a particular embodiment, the amino acid sequence of the subtilisin used in the invention is SEQ ID NO: 2.

In an embodiment, the number of amino acid substitutions, deletions and/or insertions introduced into the amino acid sequence of SEQ ID NO: 1 or SEQ ID NO: 2 is up to 10, e.g., 1, 2, 3, 4, 5, 6, 7, 8, 9, or 10; or up to 5, e.g., 1, 2, 3, 4, or 5. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for subtilisin activity to identify amino acid residues that are critical to the activity of the molecule. See also, Hilton et al., 1996, J. Biol. Chem. 271: 4699-4708. The active site of the subtilisin or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

The relatedness between two amino acid sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows: (Identical Residues×100)/(Length of Alignment−Total Number of Gaps in Alignment).

### Subtilisin Stabilizer

The subtilisin stabilizer used in the methods and compositions of the invention is a peptide aldehyde, a hydrosulfite adduct thereof, or a peptide methyl ketone. The methyl group is optionally halogen-substituted, and the peptide optionally has an N-terminal protection group.

### Peptide Aldehyde or Ketone

The stabilizer may have the formula: P-(A)_{y}-L-(B)ₓ—B⁰—R* wherein:

R* is H (hydrogen), CH₃, CX₃, CHX₂, or CH₂X. Preferably, R*═H so that the inhibitor is a peptide aldehyde with the formula P-(A)_{y}-L-(B)ₓ—B⁰—H;

X is a halogen atom, particularly F (fluorine);

B⁰ is a single amino acid residue with L- or D-configuration of the formula —NH—CH(R)—C(═O)—;

x is 1, 2 or 3;

Bₓ is independently a single amino acid residue, each connected to the next B or to B⁰ via its C-terminal;

L is absent or independently a linker group of the formula —C(═O)—, —C(═O)—C(═O)—, —C(═S)—, —C(═S)—C(═S)— or —C(═S)—C(═O)—;

A is absent if L is absent or is independently a single amino acid residue connected to L via the N-terminal of the amino acid;

P is selected from the group consisting of hydrogen or if L is absent an N-terminal protection group;

y is 0, 1, or 2,

R is independently selected from the group consisting of C₁₋₆ alkyl, C₆₋₁₀ aryl or C₇₋₁₀ arylalkyl optionally substituted with one or more, identical or different, substituent's R′;

R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂; and

R″ is a C₁₋₆ alkyl group.

x may be 1, 2 or 3 and therefore B may be 1, 2 or 3 amino acid residues respectively. Thus, B may represent B¹, B²—B¹ or B³—B²—B¹, where B³, B² and B¹ each represent one amino acid residue. y may be 0, 1 or 2 and therefore A may be absent, or 1 or 2 amino acid residues respectively having the formula A¹ or A²-A¹ wherein A² and A¹ each represent one amino acid residue.

B⁰ may be a single amino acid residue with L- or D-configuration, which is connected to H via the C-terminal of the amino acid. B⁰ has the formula —NH—CH(R)—C(═O)—, wherein R is a C₁₋₆ alkyl, C₆₋₁₀ aryl or C₇₋₁₀ arylalkyl side chain, such as methyl, ethyl, propyl, isopropyl, butyl, isobutyl, phenyl or benzyl, and wherein R may be optionally substituted with one or more, identical or different, substituent's R′. Particular examples of B⁰ are the D- or L-form of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr) and valine (Val). A particular embodiment is when B⁰ is leucine, methionine, phenylalanine, p-tyrosine and valine.

B¹, which is connected to B⁰ via the C-terminal of the amino acid, may be an aliphatic, hydrophobic and/or neutral amino acid. Examples of B¹ are alanine (Ala), cysteine (Cys), glycine (Gly), isoleucine (Ile), leucine (Leu), norleucine (Nle), norvaline (Nva), proline (Pro), serine (Ser), threonine (Thr) and valine (Val). Particular examples of B¹ are alanine, glycine, isoleucine, leucine and valine. A particular embodiment is when B¹ is alanine, glycine or valine.

If present, B², which is connected to B¹ via the C-terminal of the amino acid, may be an aliphatic, hydrophobic, neutral and/or polar amino acid. Examples of B² are alanine (Ala), arginine (Arg), capreomycidine (Cpd), cysteine (Cys), glycine (Gly), isoleucine (Ile), leucine (Leu), norleucine (Nle), norvaline (Nva), phenylalanine (Phe), proline (Pro), serine (Ser), threonine (Thr), and valine (Val). Particular examples of B² are alanine, arginine, capreomycidine, glycine, isoleucine, leucine, phenylalanine and valine. A particular embodiment is when B² is arginine, glycine, leucine, phenylalanine or valine.

B³, which if present is connected to B² via the C-terminal of the amino acid, may be a large, aliphatic, aromatic, hydrophobic and/or neutral amino acid. Examples of B³ are isoleucine (Ile), leucine (Leu), norleucine (Nle), norvaline (Nva), phenylalanine (Phe), phenylglycine, tyrosine (Tyr), tryptophan (Trp) and valine (Val). Particular examples of B³ are leucine, phenylalanine, tyrosine and tryptophan.

The linker group L may be absent or selected from the group consisting of —C(═O)—, —C(═O)—C(═O)—, —C(═S)—, —C(═S)—C(═S)— or —C(═S)—C(═O)—. Particular embodiments of the invention are when L is absent or L is a carbonyl group —C(═O)—.

A¹, which if present is connected to L via the N-terminal of the amino acid, may be an aliphatic, aromatic, hydrophobic, neutral and/or polar amino acid. Examples of A¹ are alanine (Ala), arginine (Arg), capreomycidine (Cpd), glycine (Gly), isoleucine (Ile), leucine (Leu), norleucine (Nle), norvaline (Nva), phenylalanine (Phe), threonine (Thr), tyrosine (Tyr), tryptophan (Trp) and valine (Val). Particular examples of A¹ are alanine, arginine, glycine, leucine, phenylalanine, tyrosine, tryptophan and valine. A particular embodiment is when B² is leucine, phenylalanine, tyrosine or tryptophan.

The A² residue, which if present is connected to A¹ via the N-terminal of the amino acid, may be a large, aliphatic, aromatic, hydrophobic and/or neutral amino acid. Examples of A² are arginine (Arg), isoleucine (Ile), leucine (Leu), norleucine (Nle), norvaline (Nva), phenylalanine (Phe), phenylglycine, Tyrosine (Tyr), tryptophan (Trp) and valine (Val). Particular examples of A² are phenylalanine and tyrosine.

The N-terminal protection group P (if present) may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl (Moc), (fluoromethoxy)carbonyl, benzyloxycarbonyl (Cbz), t-butyloxycarbonyl (Boc) and adamantyloxycarbonyl; p-methoxybenzyl carbonyl, benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP(OH)(═O)) and benzylphosphoramidyl (PhCH₂OP(OH)(═O)).

In the case of a tripeptide aldehyde with a protection group (i.e. x=2, L is absent and A is absent), P is preferably acetyl, methoxycarbonyl, benzyloxycarbonyl, methylamino carbonyl, methylsulfonyl, benzylsulfonyl and benzylphosphoramidyl. In the case of a tetrapeptide aldehyde with a protection group (i.e. x=3, L is absent and A is absent), P is preferably acetyl, methoxycarbonyl, methylsulfonyl, ethylsulfonyl and methylphosphoramidyl.

Suitable peptide aldehydes are described in WO94/04651, WO95/25791, WO98/13458, WO98/13459, WO98/13460, WO98/13461, WO98/13462, WO07/141736, WO07/145963, WO09/118375, WO10/055052 and WO11/036153. More particularly, the peptide aldehyde may be Cbz-Arg-Ala-Tyr-H, Ac-Gly-Ala-Tyr-H, Cbz-Gly-Ala-Tyr-H, Cbz-Gly-Ala-Tyr-CF₃, Cbz-Gly-Ala-Leu-H, Cbz-Val-Ala-Leu-H, Cbz-Val-Ala-Leu-CF₃, MeO—CO-Val-Ala-Leu-CF₃, Cbz-Gly-Ala-Phe-H, Cbz-Gly-Ala-Phe-CF₃, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO₂-Phe-Gly-Ala-Leu-H, MeSO₂-Val-Ala-Leu-H, PhCH₂O—P(OH)(O)-Val-Ala-Leu-H, EtSO₂-Phe-Gly-Ala-Leu-H, PhCH₂SO₂—Val-Ala-Leu-H, PhCH₂O—P(OH)(O)-Leu-Ala-Leu-H, PhCH₂O—P(OH)(O)-Phe-Ala-Leu-H, or MeO—P(OH)(O)-Leu-Gly-Ala-Leu-H wherein Cbz is benzyloxycarbonyl and Ac is acetyl; the preferred inhibitors for use in the liquid composition of the invention are Cbz-Gly-Ala-Tyr-H and MeO—CO-Val-Ala-Leu-H, or a hydrosulfite adduct thereof, wherein Cbz is benzyloxycarbonyl.

Further examples of such peptide aldehydes include α-MAPI, β-MAPI, Phe-C(═O)-Arg-Val-Tyr-H, Phe-C(═O)-Gly-Gly-Tyr-H, Phe-C(═O)-Gly-Ala-Phe-H, Phe-C(═O)-Gly-Ala-Tyr-H, Phe-C(═O)-Gly-Ala-L-H, Phe-C(═O)-Gly-Ala-Nva-H, Phe-C(═O)-Gly-Ala-Nle-H, Tyr-C(═O)-Arg-Val-Tyr-H, Tyr-C(═O)-Gly-Ala-Tyr-H, Phe-C(═S)-Arg-Val-Phe-H, Phe-C(═S)-Arg-Val-Tyr-H, Phe-C(═S)-Gly-Ala-Tyr-H, Antipain, GE20372A, GE20372B, Chymostatin A, Chymostatin B, and Chymostatin C.

### Hydrosulfite Adduct

The subtilisin stabilizer may be a hydrosulfite adduct of the peptide aldehyde described above, e.g. as described in WO 2013/004636. The adduct may have the formula P-(A)_{y}-L-(B)ₓ—N(H)—CHR—CH(OH)—SO₃M, wherein P, A, y, L, B, x and R are defined as above, and M is H or an alkali metal, preferably Na or K. A preferred embodiment is a hydrosulfite adduct wherein P=Cbz, B²=Gly; B¹=Ala; B⁰=Tyr (so R=PhCH₂, R′═OH), x=2, y=0, L=A=absent and M=Na.

### Peptide Aldehyde or Hydrosulfite Adduct

The stabilizer may be an aldehyde having the formula P—B²—B¹—B⁰—H or an adduct having the formula P—B²—B¹—N(H)—CHR—CHOH—SO₃M, wherein

a) H is hydrogen;
b) B⁰ is a single amino acid residue with L- or D-configuration of the formula —NH—CH(R)—C(═O)—;
c) B¹ and B² are independently single amino acid residues;
d) R is independently selected from the group consisting of C₁₋₆ alkyl, C₆₋₁₀ aryl or C₇₋₁₀ arylalkyl optionally substituted with one or more, identical or different, substituent's R′;
e) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
f) R″ is a C₁₋₆ alkyl group; and
g) P is an N-terminal protection group.
Constituents b) to g) may be selected as described above.

### Peroxide

The liquid compositions of the invention comprise a peroxide or a peroxide source. In an embodiment, the composition may comprise a peroxide or peroxide source chosen from organic and inorganic peroxides, such as hydrogen peroxide, peroxy acids (e.g., peracetic acid and higher alkyl peracids), alkylhydroperoxides, dialkylperoxides, urea-hydrogen peroxide, inorganic perhydrate salts (e.g., sodium salts of perborate, including mono- or tetrahydrate perborate salts), percarbonates, persulfates, perphosphates, persilicates, and combinations thereof.

In an embodiment, the composition comprises a peroxide or peroxide source in an amount ranging from about 1 ppm to about 50% by weight of the total composition. Preferably, the peroxide or peroxide source is present in an amount ranging from about 5 ppm to about 15%, or from about 10 ppm to about 10%, or from about 0.01% to about 10%, or from about 0.1% to about 10% by weight of the total composition.

The liquid compositions of the invention may optionally comprise a peroxide stabilizer. The purpose of the peroxide stabilizer is to reduce the rate of peroxide decomposition during storage of the liquid compositions of the invention. The peroxide stabilizer may also exhibit a stabilizing effect on the subtilisin.

Examples of peroxide stabilizers include, but are not limited to, stannates, phosphates, pyrophosphates, carboxylates, organic chelating agents, and combinations thereof. Suitable stabilizers may include stannates, for example, such as stannic chloride, stannic oxide, stannic bromide, stannic chromate, stannic iodide, stannic sulfide, tin dichloride bis(2, 4-pentanedionate), tin phthalocyanine dichloride, tin acetate, and the like. The liquid compositions of the invention may also comprise additional stabilizers, such as aromatic chelating agents or aromatic radical scavengers, known to one of ordinary skill in the art. Specific examples of peroxide stabilizers that may be used in accordance with the present invention include, but are not limited to, sodium stannate, potassium stannate, ethylenediaminetetraacetic acid (EDTA), amine-substituted organophosphonic acids or their salts, butylated hydroxytoluene (BHT), butylated hydroxyanisole (BHA), phenols, and combinations thereof.

The peroxide stabilizer may also be selected from carboxylic acid/or carboxylic acid salt, and certain soluble polymers.

The carboxylic acid or carboxylate salt may comprise a short aliphatic chain of 12 carbon atoms or less or have an aromatic group, such as a phenyl group or hydroxyphenyl group. In at least one embodiment, the enzyme protection agent is chosen from acetic acid, benzoic acid, picolinic acid, salicylic acid, or sodium salicylate.

The soluble polymers may be selected from the group consisting of polyacrylates, polymethacrylates, polyethoxylates, polyacrylamide, polyquaterniums, and polybetaines. In at least one embodiment, the polymer is chosen from polyquaternium-11, polyquaternium-16, polyDADMAC, poly (acrylamido-N-propyltrimethylammonium chloride) (polyATPAC), polyoxypropylene-polyoxyethylene block copolymers (e.g., Pluronic® 25R2), polyethylene glycols (e.g., PEG-40 stearate, PEG 8000), polyacrylates (e.g., Acusol® 425N), and poly(3-(3-acrylamidopropyldimethylammonio)propionate) (polyAMDAP). In at least one embodiment, the polymer is a cationic polymer, such as, for example, a polyquaternium, such as polyDADMAC and Luviquat® PQ 11.

The compositions of the invention may contain a peroxide stabilizer in an amount ranging from about 10 ppm to about 30% by weight of the total composition. Preferably, the peroxide stabilizer is present in an amount ranging from about 0.01% to about 10%, such as, from about 0.01% to about 5%, or from about 0.01% to about 1% by weight of the total composition.

### Enzymes

Other non-subtilisin enzymes which can be added to the compositions of the invention, may be one or more enzymes selected from the group consisting of lipase, cutinase, amylase, carbohydrase, cellulase, pectinase, pectate lyase, mannanase, arabinase, galactanase, xylanase, DNAse, perhydrolase, and oxidoreductase (oxidase, laccase, peroxidase, haloperoxidase).

The methods and compositions of the invention may include 1, 2, 3, 4, 5, 6, 7, or 8 non-subtilisin enzyme(s). A non-subtilisin enzyme is an enzyme, preferably a detergent enzyme, which is not a subtilisin.

### Lipase/Cutinase

Suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258068 and EP 305216, cutinase from Humicola, e.g. H. insolens as described in WO 96/13580, a Pseudomonas lipase, e.g., from P. alcaligenes or P. pseudoalcaligenes (EP 218272), P. cepacia (EP 331376), P. stutzeri (GB 1372034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., Biochemica et Biophysica Acta, (1993), 1131, 253-360), B. stearothermophilus (JP 64/744992) or B. pumilus (WO 91/16422), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in WO 92/05249, WO 94/01541, EP 407225, EP 260105, WO 95/35381, WO 96/00292, WO 95/30744, WO 94/25578, WO 95/14783, WO 95/22615, WO 97/04079, WO 97/07202, WO 00/060063, WO 07/087508 and WO 09/109500.

Preferred commercially available lipase enzymes include Lipolase™, Lipolase Ultra™′ and Lipex™, Lipex Evity™, Lecitase™, Lipolex™; Lipoclean™, Lipoprime™ (Novozymes A/S). Other commercially available lipases include Lumafast (Genencor Int Inc); Lipomax (Gist-Brocades/Genencor Int Inc) and Bacillus sp lipase from Solvay.

### Carbohydrase

A carbohydrase is a general term for enzymes that cleave carbohydrates. In general carbohydrases are named after the substrates they act on, for example amylases act on amylase and cellulases act on cellulose. Many carbohydrases have found use in cleaning and laundry applications, such as amylase, cellulase, pectinase, pectate lyase, mannanase, arabinase, galactanase and xylanase, and all these can be applied in the liquid composition.

### Amylase

Suitable amylases include those of bacterial or fungal origin. Chemically modified or protein engineered mutants are included. Amylases include, for example, α-amylases obtained from Bacillus, e.g., a special strain of Bacillus licheniformis, described in more detail in GB 1,296,839.

Examples of suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193. Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

### M197T;

### H156Y+A181T+N190F+A209V+Q264S; or

### G48A+T491+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

### N128C+K178L+T182G+Y305R+G475K;

### N128C+K178L+T182G+F202Y+Y305R+D319T+G475K;

### S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Stainzyme™ Stainzyme Plus™ Amplify™, Amplify Prime™, Resilience™, Everest™, Duramyl™, Termamyl™, Termamyl Ultra™, Natalase™, Fungamyl™ and BAN™ (Novozymes A/S), Rapidase™ and Purastar™/Effectenz™, Powerase™ and Preferenz S100 (from Genencor International Inc./DuPont).

### Lyases

The lyase may be a pectate lyase derived from Bacillus, particularly B. lichemiformis or B. agaradhaerens, or a variant derived of any of these, e.g. as described in U.S. Pat. No. 6,124,127, WO 99/027083, WO 99/027084, WO 02/006442, WO 02/092741, WO 03/095638, Commercially available pectate lyases are XPect™, Pectawash™, and Pectaway™ (Novozymes A/S).

### Mannanase

The mannanase may be an alkaline mannanase of Family 5 or 26. It belongs It may be a wild-type from Bacillus or Humicola, particularly B. agaradhaerens, B. licheniformis, B. halodurans, B. clausii, or H. insolens. Suitable mannanases are described in WO 99/064619. A commercially available mannanase is Mannaway™ (Novozymes A/S).

### Cellulase

Suitable cellulases may be of bacterial or fungal origin. Chemically or genetically modified mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593.

Commercially available cellulases include Carezyme™, Celluzyme™, Celluclean™, Celluclast™, Endolase™, Renozyme™, Carezyme Premium™, Whitezyme™ (Novozymes A/S); Clazinase™, Puradax, Puradax HA, and Puradax EG (available from Genencor); and KAC-500(B)™ (Kao Corporation), Biotouch™ (AB enzymes).

### Peroxidases/Oxidases

Suitable peroxidases are comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

The peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus. A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Deoxyribonuclease (DNase)

Suitable deoxyribonucleases (DNases) are any enzyme that catalyzes the hydrolytic cleavage of phosphodiester linkages in the DNA backbone, thus degrading DNA. According to the invention, a DNase which is obtainable from a bacterium is preferred; in particular a DNase which is obtainable from a Bacillus is preferred; in particular a DNase which is obtainable from Bacillus subtilis or Bacillus licheniformis is preferred. Examples of such DNases are described in patent application WO 2011/098579 or in PCT/EP2013/075922.

### Perhydrolase

Suitable perhydrolases are capable of catalyzing a perhydrolysis reaction that results in the production of a peracid from a carboxylic acid ester (acyl) substrate in the presence of a source of peroxygen (e.g., hydrogen peroxide). While many enzymes perform this reaction at low levels, perhydrolases exhibit a high perhydrolysis:hydrolysis ratio, often greater than 1. Suitable perhydrolases may be of plant, bacterial or fungal origin. Chemically modified or protein engineered mutants are included.

Examples of useful perhydrolases include naturally occurring Mycobacterium perhydrolase enzymes, or variants thereof. An exemplary enzyme is derived from Mycobacterium smegmatis. Such enzyme, its enzymatic properties, its structure, and variants thereof, are described in WO 2005/056782, WO 2008/063400, US 2008/145353, and US2007167344.

### Liquid Detergent Composition

The liquid detergent composition has a physical form, which is not solid (or gas). It may be a pourable liquid, a pourable gel or a non-pourable gel. It may be either isotropic or structured, preferably isotropic. It may be a formulation useful for washing in automatic washing machines or for hand washing.

The liquid detergent composition may be aqueous, typically containing at least 20% by weight and up to 95% water, such as up to 70% water, up to 50% water, up to 40% water, up to 30% water, or up to 20% water. Other types of liquids, including without limitation, alkanols, amines, diols, ethers and polyols may be included in an aqueous liquid detergent. An aqueous liquid detergent may contain from 0-30% organic solvent. A liquid detergent may even be non-aqueous, wherein the water content is below 10%, preferably below 5%.

Detergent ingredients can be separated physically from each other by compartments in water dissolvable pouches. Thereby negative storage interaction between components can be avoided. Different dissolution profiles of each of the compartments can also give rise to delayed dissolution of selected components in the wash solution.

The detergent composition may take the form of a unit dose product. A unit dose product is the packaging of a single dose in a non-reusable container. It is increasingly used in detergents for laundry and dish wash. A detergent unit dose product is the packaging (e.g., in a pouch made from a water soluble film) of the amount of detergent used for a single wash.

Pouches can be of any form, shape and material which is suitable for holding the composition, e.g., without allowing the release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be a blend compositions comprising hydrolytically degradable and water soluble polymer blends such as polyactide and polyvinyl alcohol (known under the Trade reference M8630 as sold by Chris Craft In. Prod. Of Gary, Ind., US) plus plasticizers like glycerol, ethylene glycerol, Propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids (see e.g., US 2009/0011970).

The choice of detergent components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The choice of additional components is within the skill of the artisan and includes conventional ingredients, including the exemplary non-limiting components set forth below.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 0.1% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually contain from about 0.1% to about 20% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, betaines, e.g. cocamidopropyl betaine; and combinations thereof.

When included therein the detergent will usually contain from about 0.1% to about 10% by weight of a zwitterionic surfactant. Non-limiting examples of zwitterionic surfactants include betaine, alkyldimethylbetaine, sulfobetaine, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see for example review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SOS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 1% to about 50% of a detergent builder or co-builder, or a mixture thereof. In an automatic dish wash (ADVV) detergent, the level of builder is typically 40-65%, particularly 50-65%. In a liquid laundry detergent or a prespotter detergent, the level of detergent builder or co-builder is typically below 10%. The builder and/or co-builder may particularly be a chelating agent that forms water-soluble complexes with Ca and Mg ions. Any builder and/or co-builder known in the art for use in laundry detergents may be utilized. Non-limiting examples of builders include citrates, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-50% by weight, such as about 0.5% to about 10%, of a detergent co-builder, or a mixture thereof. The detergent composition may include include a co-builder alone, or in combination with a builder, for example a citrate builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575 and U.S. Pat. No. 5,955,415. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g., WO 2007/087257 and WO 2007/087243.

### Additional Enzymes

The liquid detergent composition may comprise additional enzymes using other formulation technologies, such as microcapsules (e.g., as described in PCT/EP2014/059017 or WO 1997/024177), particles, or enzymatic water soluble films (e.g., as described in PCT/US2014/027603, PCT/US2014/027462, or WO 2013/148492).

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants—

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent—

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Soil Release Polymers—

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents—

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Rheology Modifiers are structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of the composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Bleaching Systems

Due to the incompatibility—prior to the present invention—of the components there are still only few examples of liquid detergents combining bleach and enzymes (e.g., U.S. Pat. No. 5,275,753 or WO 99/00478). The detergent may contain 0-50% of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 98/17767. A particular family of bleach activators of interest was disclosed in EP624154 and particularly preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

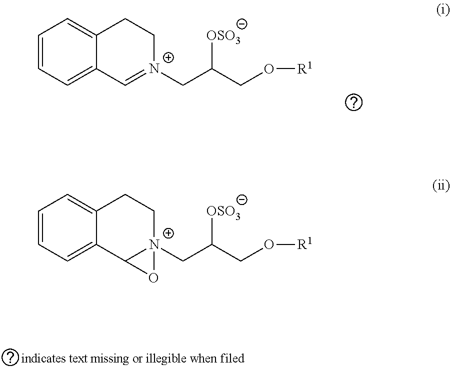

and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g., in WO 2007/087258, WO 2007/087244, WO 2007/087259 and WO 2007/087242. Suitable photobleaches may for example be sulfonated zinc phthalocyanine or sulfonated tetrabenzo-tetraazaporphine derivatives (Tinolux BMC/BSS/BSR, BASF).

### Formulation of Detergent Products

The liquid detergent composition of the invention may be in any convenient form, e.g., a pouch having one or more compartments, a gel, or a regular, compact or concentrated liquid detergent (see e.g., WO 2009/098660 or WO 2010/141301).

Pouches can be configured as single or multi compartments. It can be of any form, shape and material which is suitable for holding the composition, e.g., without allowing release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Ind., USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids.

Detergent ingredients can be separated physically from each other by compartments in water dissolvable pouches. Thereby negative storage interaction between components can be avoided. Different dissolution profiles of each of the compartments can also give rise to delayed dissolution of selected components in the wash solution.

### Compositions, Methods and Uses

As described in the above paragraphs, the present invention provides a liquid composition comprising:

(a) a peroxide or peroxide source,
(b) a subtilisin having an amino acid sequence comprising a substitution corresponding to M216 of SEQ ID NO: 1,
(c) a peptide aldehyde or ketone subtilisin stabilizer, or a hydrosulfite adduct thereof, and
(d) optionally a peroxide stabilizer.

In an embodiment, the liquid composition maintains at least 10% of the enzyme activity (i.e., the residual enzyme activity) after 4 weeks at 37° C. Preferably, at least 10%, at least 15%, at least 20%, at least 25%, at least 30%, at least 35%, or at least 40% of the enzyme activity remains after 4 weeks at 37° C.; or at least 10%, at least 15%, at least 20%, at least 25%, at least 30%, at least 35%, or at least 40% of the enzyme activity remains after 4 weeks at 30° C.

In an embodiment, the liquid composition is a homogeneous, single-phase solution. In another embodiment, the liquid composition may comprise a solid phase. For example, a portion of certain components of the liquid composition may precipitate out of solution or the liquid composition may comprise a solid phase component, such as, for example, a suspended peroxide. However, in these liquid compositions comprising a solid content, the liquid component comprises at least a portion of the subtilisin and the peroxide or peroxide source in the liquid phase. In an embodiment, at least 50% of the enzyme and peroxide content is in the liquid phase, such as, for example, at least 75%, at least 90%, or at least 95% of the total amount of enzyme and peroxide in the composition.

In an embodiment, the compositions comprise water in an amount of at least 20% by weight of the total composition. Preferably, water is present in the composition in an amount of at least 35%, at least 50%, at least 75%, or at least 80% by weight of the total composition.

The liquid compositions of the present invention may be formulated as cleaning solutions. Exemplary cleaning solutions include, but are not limited to, laundry detergent, fabric softener, laundry prespotter (spray or gel or pen), auxiliary bleach (liquid or paste), hand dish detergent, automatic dishwasher detergent (gel or paste or suspension), carpet prespotter, carpet cleaner, hard surface cleaner (spray or concentrated/dilutable), toilet bowl cleaner, hand detergent, general basin/tub/tile foam cleaner, abrasive surface cleaner, medical cleaner, or disinfection cleaning solutions.

Liquid compositions according to the invention may also be formulated for use in the following applications:
Pulp and paper: bleaching, brightening, and delignification in mechanical and chemical pulping, and deinking during paper recycling;
Personal care: antiseptic applications, hair bleaching and coloring, tooth whitening and oral care;
Chemical processes: general oxidation reactions including but not limited to epoxidation, hydroxylation, bromine reactivation, organic peroxide production, amine oxidation, processes for chemical or pharmaceutical synthesis or manufacture, as well as decolorization;
Textile or fiber bleaching;
Environmental: water treatment, wastewater or storm water treatment, including but not limited to pollutant degradation and decolorization, and wastewater or storm water odor reduction or elimination;
General broad-spectrum disinfection and sanitization, mold/mildew, spore, virus, fungus removers;
Defense: chemical or biological warfare agent degradation;
Improved delignification for increased cellulosic ethanol production or for the production of useful organic chemicals from biomass; and
Desulfurization of diesel fuel, gasoline, kerosene, biodiesel, coal, or natural gas.

The invention also provides for use of a peptide aldehyde or ketone subtilisin stabilizer, or a hydrosulfite adduct thereof, for improving the stability in a liquid hydrogen peroxide composition of a subtilisin having an amino acid sequence comprising a substitution in a position corresponding to M216 of SEQ ID NO: 1.

The pH of the liquid composition may be in the range 5.0-11; preferably in the range 5.5-10; more preferably in the range 6.0-9.5; or particularly in the range 7-9. pH may be measured directly in the composition or in a 5% solution in water.

### EXAMPLES

Chemicals used as buffers and substrates were commercial products of at least reagent grade.
Subtilisin1 has the amino acid sequence as shown in SEQ ID NO: 2, which is a subtilisin having a M216 substitution according to the invention.
Subtilisin2 has the amino acid sequence as shown in SEQ ID NO: 1, which is a reference subtilisin not covered by the invention.
The stock solution of each subtilisin contains 4.4% w/w of active enzyme protein.
Stabilizer1: Cbz-Gly-Ala-Tyr-H (C-terminal aldehyde), wherein Cbz is benzyloxycarbonyl; which is a subtilisin stabilizer according to the invention. When used in the examples below, Stabilizer1 was added to the subtilisin stock solution at a concentration of 0.28% w/w.
Stabilizer2: Cbz-Gly-Ala-NHCH(CH₂C₆H₄pOH)CH(OH)(OSO3)Na (hydrosulfite adduct of a C-terminal aldehyde) wherein Cbz is benzyloxycarbonyl; which is a subtilisin stabilizer according to the invention. When used in the examples below, Stabilizer2 was added to the subtilisin stock solution at a concentration of 0.35% w/w.
Stabilizer3: MeOCO-Val-Ala-Leu-H (C-terminal aldehyde). When used in the examples below, Stabilizer3 was added to the subtilisin stock solution at a concentration of 0.28% w/w.
Stabilizer4: 4-formyl-phenyl-boronic acid (4-FPBA); which is a reference protease stabilizer not covered by the invention. When used in the examples below, Stabilizer4 was added to the subtilisin stock solution at a concentration of 3.4% w/w.

The compositions were prepared as shown in the examples and samples stored in closed vials for storage. Residual activity was measured by comparing activity in a stored sample at the time of taken out of incubation without freezing and comparing activity to a freshly prepared sample without peroxide and dividing activity in the stored sample with activity in the fresh sample.

Activity analysis is performed by analysing protease activity by hydrolysis of N,N-dimethylcasein to produce a carboxylic acid and a primary amine compounds. The primary amines that are formed react, under alkaline conditions, with tri-nitrobenzenesulfonic acid (TNBS) to form a colored complex. The amount of colored complex that forms over the fixed reaction period at a constant temperature (530 seconds at 40° C.; complex absorbance is measured at 405 nm) is related to the amount of active protease present in the sample. The amount of colored product complex produced is compared to a standard curve based on known protease concentrations to calculate the amount of active protease in the detergent sample. The amount of protease activity at a specific time point is compared to the t=0 activity level to calculate the % active protease remaining.

Residual hydrogen peroxide (H₂O₂) was measured by permanganate titration (reduction of potassium permanganate by hydrogen peroxide in sulfuric acid).

### Example 1

### Subtilisin Stability in Liquid Peroxide Compositions

TABLE 1
Components in the liquid peroxide composition

| Component | % w/w |
|---|---|
| CaNO₃ | 1.6 |
| Sorbitol | 5.0 |
| H₂O₂ | 0.8 |
| Sodium salicylate | 0.017 |
| polyDADMAC polymer | 0.1 |
| Subtilisin1 or Subtilisin2 | 1.0 |
| stock solution | |
| Water | ad 100% |
| pH | 7.0 |

TABLE 2
Stability of Subtilisin1 and Subtilisin2 in the
liquid peroxide composition in Table 1.

% Subtilisin residual
activity after 4 weeks

| Storage Temperature | Subtilisin1 | Subtilisin2 |
|---|---|---|
| 22° C. | 81% | 35% |
| 30° C. | 61% | 33% |
| 37° C. | 46% | 15% |

The results in Table 2 show that the stability of Subtilisin1 is much better than that observed for Subtilisin2 in the liquid peroxide composition in Table 1.

TABLE 3
Residual H₂O₂ in the liquid peroxide composition
in Table 1 after storage.

Residual H₂O₂ after
4 weeks

| Storage Temperature | Subtilisin1 | Subtilisin2 |
|---|---|---|
| 22° C. | 99.0% | — |
| 30° C. | 98.7% | — |
| 37° C. | 97.8% | 95.8% |

The stability results in Table 3 show that the active level of hydrogen peroxide in both systems is similar, and that the significant difference in enzyme stability of Subtilisin1 and Subtilisin1 is not caused by different peroxide contents.

### Example 2

### Benefit of a Subtilisin Stabilizer in a Liquid Peroxide Composition

This study examined Subtilisin1 stability in the presence of hydrogen peroxide, with and without subtilisin stabilizers (Stabilizer4 and Stabilizer2) in two different model detergents at pH 7 and 9.

Several examples of the effect and benefit of Subtilisin1 stabilization using Stabilizer2 is shown in the Tables below. Stabilizer4 was included as a reference for comparison. It is clear that Stabilizer2 provides much more effective stabilization compared to Stabilizer4, even though both are known as subtilisin inhibitors. Surprisingly, Stabilizer2 improves stabilization of Subtilisin1 even more in some of the higher pH formulations.

### Nonionic Pretreatment Model Detergent

TABLE 4
Liquid nonionic surfactant based pretreatment model detergent system

| Component | % w/w |
|---|---|
| Glycerol | 3.0 |
| AE (Biosoft N25-9) | 5.0 |
| CaCl₂•2H₂O | 0.1 |
| H₂O₂ | 1.0 |
| Sodium salicylate | 0.017 |
| polyDADMAC polymer | 0.1 |
| Subtilisin1 stock solution with | 1.0 |
| Stabilizer2, Stabilizer4, or no | |
| stabilizer | |
| Water | ad 100% |
| pH | 7 or 9 |

TABLE 5
Residual activity of Subtilisin1 after storage in the liquid
nonionic pretreatment detergent in Table 4.

| | | |
|---|---|---|
| Subtilisin 1 + | Subtilisin 1 + | Subtilisin 1 + |
| no stabilizer | Stabilizer 4 | Stabilizer 2 |

| Temp. | pH 7 | pH 9 | pH 7 | pH 9 | pH 7 | pH 9 |
|---|---|---|---|---|---|---|
| 22° C. | 85% | 40% | 64% | 49% | 95% | 103% |
| 30° C. | 65% | 19% | 44% | 27% | 86% | 72% |
| 37° C. | 44% | 12% | 26% | 21% | 55% | 36% |

TABLE 6
Residual* H₂O₂ in the nonionic pretreatment
detergent in Table 4 (after storage).

| | Subtilisin 1 + | | Subtilisin 1 + | | Subtilisin 1 + | |
|---|---|---|---|---|---|---|
| Storage | no stabilizer | | 4-FPBA | | Z-GAY-H | |
| temperature | pH 7 | pH 9 | pH 7 | pH 9 | pH 7 | pH 9 |
| 22° C. | ≥100% | 93% | ≥100% | ≥100% | ≥100% | 84% |
| 30° C. | ≥100% | 91% | — | ≥100% | ≥100% | 84% |
| 37° C. | ≥100% | 90% | ≥100% | ≥100% | ≥100% | 79% |

*relative to the initially added hydrogen peroxide level = 1%

### Nonionic Laundry Model Detergent

TABLE 7
Liquid nonionic surfactant based laundry model detergent system

| Component | % w/w |
|---|---|
| Glycerol | 5.0 |
| AE (Biosoft N25-9) | 15.0 |
| CaCl₂•2H₂O | 0.1 |
| H₂O₂ | 1.0 |
| Sodium salicylate | 0.017 |
| polyDADMAC polymer | 0.1 |
| Subtilisin1 stock solution with | 1.0 |
| Stabilizer2, Stabilizer4, or no | |
| stabilizer | |
| Water | ad 100% |
| pH | 7 or 9 |

TABLE 8
Residual activity of Subtilisin1 after storage in the liquid
nonionic laundry detergent in Table 7.

| | Subtilisin 1 + | | Subtilisin 1 + | | Subtilisin 1 + | |
|---|---|---|---|---|---|---|
| Storage | no stabilizer | | Stabilizer4 | | Stabilizer2 | |
| temperature | pH 7 | pH 9 | pH 7 | pH 9 | pH 7 | pH 9 |
| 22° C. | 63% | 75% | 61% | 42% | 86% | 85% |
| 30° C. | 47% | 68% | 48% | 29% | 78% | 77% |
| 37° C. | 30% | 45% | 22% | 17% | 54% | 56% |

TABLE 9
Residual* H₂O₂ in the nonionic laundry
detergent in Table 7 (after storage).

| | Subtilisin 1 + | | Subtilisin 1 + | | Subtilisin 1 + | |
|---|---|---|---|---|---|---|
| Storage | no stabilizer | | Stabilizer 4 | | Stabilizer 2 | |
| temperature | pH 7 | pH 9 | pH 7 | pH 9 | pH 7 | pH 9 |
| 22° C. | 95% | 95% | 96% | 95% | 95% | 95% |
| 30° C. | 94% | 94% | 100% | 93% | 94% | 91% |
| 37° C. | 92% | 91% | 92% | 92% | — | 90% |

*relative to the initially added hydrogen peroxide level = 1%

### Nonionic/Anionic Pretreatment Detergent

TABLE 10
Liquid nonionic/anionic surfactant based pretreatment
model detergent system.

| Component | % w/w |
|---|---|
| Glycerol | 3.0 |
| AE (Biosoft N25-9) | 5.0 |
| AES (Steol CS 370) | 0.7 |
| CaCl₂•2H₂O | 0.1 |
| H₂O₂ | 1.0 |
| Subtilisin1 stock solution with | 1.0 |
| Stabilizer2, Stabilizer4, or no | |
| stabilizer | |
| Water | ad 100% |
| pH | 7 or 9 |

TABLE 11
Residual activity of Subtilisin1 after storage in the liquid
nonionic/anionic pretreatment detergent in Table 10.

| | Subtilisin1 + | | Subtilisin1 + | | Subtilisin1 + | |
|---|---|---|---|---|---|---|
| Storage | no stabilizer | | Stabilizer 4 | | Stabilizer 2 | |
| temperature | pH 7 | pH 9 | pH 7 | pH 9 | pH 7 | pH 9 |
| 22° C. | 79% | 57% | 69% | 62% | 84% | 89% |
| 30° C. | 66% | 47% | 49% | 35% | 90% | 83% |
| 37° C. | 52% | 29% | 32% | 19% | 72% | 69% |

TABLE 12
Residual* H₂O₂ in the nonionic/anionic
pretreatment detergent in Table 10 (after storage).

| | Subtilisin 1 + | | Subtilisin 1 + | | Subtilisin 1 + | |
|---|---|---|---|---|---|---|
| Storage | no stabilizer | | Stabilizer 4 | | Stabilizer 2 | |
| temperature | pH 7 | pH 9 | pH 7 | pH 9 | pH 7 | pH 9 |
| 22° C. | 88% | — | 94% | 84% | 90% | — |
| 30° C. | 86% | — | 92% | 81% | 97% | — |
| 37° C. | 100% | 95% | 92% | 77% | 90% | — |

*relative to the initially added hydrogen peroxide level = 1%

### Nonionic/Anionic Laundry Model Detergent

TABLE 13
Liquid nonionic/anionic surfactant based laundry
model detergent system.

| Component | % w/w |
|---|---|
| Glycerol | 5.0 |
| AE (Biosoft N25-9) | 13.0 |
| AES (Steol CS 370) | 2.0 |
| CaCl₂•2H₂O | 0.1 |
| H₂O₂ | 1.0 |
| Sodium salicylate | 0.017 |
| polyDADMAC polymer | 0.1 |
| Subtilisin1 stock solution with | 1.0 |
| Stabilizer2, Stabilizer4, or no | |
| stabilizer | |
| Water | ad 100% |
| pH | 7 or 9 |

TABLE 14
Residual activity of Subtilisin1 after storage in the liquid
nonionic/anionic laundry detergent in Table 13.

| | Subtilisin1 + | | Subtilisin 1 + | | Subtilisin 1 + | |
|---|---|---|---|---|---|---|
| Storage | no stabilizer | | Stabilizer 4 | | Stabilizer 2 | |
| temperature | pH 7 | pH 9 | pH 7 | pH 9 | pH 7 | pH 9 |
| 22° C. | 84% | 63% | 94% | 51% | 70% | 90% |
| 30° C. | 62% | 34% | 68% | 26% | 64% | 82% |
| 37° C. | 40% | 23% | 40% | 11% | 47% | 46% |

TABLE 15
Residual* H₂O₂ in the nonionic/anionic
laundry detergent in Table 13 (after storage).

| | Subtilisin 1 + | | Subtilisin 1 + | | Subtilisin 1 + | |
|---|---|---|---|---|---|---|
| Storage | no stabilizer | | Stabilizer 4 | | Stabilizer 2 | |
| temperature | pH 7 | pH 9 | pH 7 | pH 9 | pH 7 | pH 9 |
| 22° C. | 101% | 95% | 98% | 96% | 97% | 92% |
| 30° C. | 99% | 92% | 107% | 92% | 97% | 93% |
| 37° C. | 101% | 97% | 109% | 90% | — | 91% |

*relative to the initially added hydrogen peroxide level = 1%

### 2% Hydrogen Peroxide System—Nonionic/Anionic Pretreatment Model Detergent

TABLE 16
Liquid nonionic/anionic surfactant based pretreatment
model detergent system.

| Component | % w/w |
|---|---|
| Glycerol | 3.0 |
| AE (Biosoft N25-9) | 5.0 |
| AES (Steol CS 370) | 0.7 |
| CaCl₂•2H₂O | 0.1 |
| H₂O₂ | 2.0 |
| Subtilisin1 stock solution with | 1.0 |
| Stabilizer2, Stabilizer4, or no | |
| stabilizer | |
| Water | ad 100% |
| pH | 7 or 9 |

TABLE 17
Residual activity of Subtilisin1 after storage in the liquid
nonionic/anionic pretreatment detergent in Table 16.

| | Subtilisin 1 + | | Subtilisin 1 + | | Subtilisin 1 + | |
|---|---|---|---|---|---|---|
| Storage | no stabilizer | | Stabilizer 4 | | Stabilizer 2 | |
| temperature | pH 7 | pH 9 | pH 7 | pH 9 | pH 7 | pH 9 |
| 22° C. | 85% | 30% | 71% | 61% | 83% | 70% |
| 30° C. | 69% | 12% | 31% | 22% | 78% | 50% |
| 37° C. | 50% | 5% | 23% | 17% | 61% | 20% |

TABLE 18
Residual* H₂O₂ in the nonionic/anionic
pretreatment detergent in Table 16 (after storage).

| | Subtilisin 1 + | | Subtilisin 1 + | | Subtilisin 1 + | |
|---|---|---|---|---|---|---|
| Storage | no stabilizer | | Stabilizer 4 | | Stabilizer 2 | |
| temperature | pH 7 | pH 9 | pH 7 | pH 9 | pH 7 | pH 9 |
| 22° C. | 87% | — | 93% | 87% | 88% | 73% |
| 30° C. | 84% | — | 83% | 82% | 85% | 62% |
| 37° C. | 90% | 92% | 88% | 84% | 88% | 56% |

*relative to the initially added hydrogen peroxide level = 2%

### Example 3

### Comparative Study of Different Stabilizers and Subtilisin1 Illustrating the Benefits of the Claimed Protease Stabilizers.

TABLE 19

| | “NI pretreat” | “Mix Laundry” |
|---|---|---|
| Component | (% w/w) | (% w/w) |
| Glycerol | 3.0 | 5.0 |
| AEO (C12-15 7EO) | 5.0 | 10.0 |
| SLES | — | 5.0 |
| LAS | — | 5.0 |
| CaCl₂ | 0.06 | 0.06 |
| Na-citrate | 1.0 | 1.0 |
| H₂O₂ | +/−1.0 | +/−1.0 |
| Subtilisin1 with Stabilizer1, | 1.0 | 1.0 |
| Stabilizer2, Stabilizer3, or Stabilizer4 | | |
| Borax | +/−1.0 | +/−1.0 |
| Sodium salicylate | +/−0.017 | +/−0.017 |
| polyDADMAC polymer | +/−0.1 | +/−0.1 |
| Water | ad 100 | Ad 100 |
| pH | 7.0 | 7.5 |

### Protease Stability Results

TABLE 20
Residual activity +/−1% H₂O₂ after storage for 2 weeks at 37° C.

| | “NI pretreat” | “NI pretreat” |
|---|---|---|
| | without H₂O₂ | with 1% H₂O₂ |
| No stabilizer | 82% | 38% |
| Stabilizer1 | 121% | 86% |
| Stabilizer2 | 98% | 61% |
| Stabilizer3 | 95% | 64% |
| Stabilizer4 | 129% | 11% |
| 1% borax | 117% | 22% |
| Others | no sodium salicylate | with sodium salicylate |
| | no polyDADMAC | with polyDADMAC |

### Protease Stability Results

TABLE 21
Residual activity +/−1% H2O2 after storage for 2 weeks at 30° C.

| | “Mix Laundry” | “Mix Laundry” |
|---|---|---|
| | no H₂O₂ | with 1% H₂O₂ |
| No stabilizer | 73% | 42% |
| Stabilizer1 | 97% | 66% |
| Stabilizer2 | 83% | 52% |
| Stabilizer3 | 83% | 60% |
| Stabilizer4 | 86% | 20% |
| 1% borax | 81% | 43% |
| Others | no sodium salicylate | with sodium salicylate |
| | no polyDADMAC | with polyDADMAC |

### Example 4

The detergent composition of the invention could also be a manual dish wash detergent as shown in Table 22.

TABLE 22
Manual dish wash detergent composition.

| Component | % w/w | % w/w |
|---|---|---|
| Glycerol | 5.0 | 1.0 |
| AEO (C12-15 7EO) | 5.0 | 1.0 |
| SLES | 15.0 | 5.0 |
| Amine Oxide | 5.0 | 2.0 |
| LAS | — | 2.0 |
| Na-citrate | 1.0 | 0.3 |
| H₂O₂ | 1.0 | 0.5 |
| Subtilisin1 with Stabilizer1, | 1.0 | 0.5 |
| Stabilizer2, Stabilizer3, or | | |
| Stabilizer4 | | |
| Sodium salicylate | +/−0.017 | +/−0.017 |
| polyDADMAC polymer | +/−0.1 | +/−0.1 |
| NaCl | — | 0.5 |
| Ethanol | — | 2.0 |
| Water | ad 100 | Ad 100 |
| pH | 5.5 | 7.0 |

The (manual dish wash) detergent composition could also comprise the ingredients listed below.

### Detergent Composition 1

Water; sodium laureth sulfate; lauramine oxide; alcohol; ppg-34; sodium chloride; 2-propylheptanol ethoxylated; phenoxyethanol; sodium hydroxide; parfum; dipropylene glycol; benzisothiazolinone; sodium bicarbonate; sodium diethylenetriamine pentamethylene phosphonate; dimethyl lauramine; colorants; dimethyl myristamine; sodium carbonate; pentasodium pentetate; dimethyl palmitamine; hydrogen peroxide; sodium chlorate; methyl alcohol; protease; protease stabilizer of the invention.

### Detergent Composition 2

Water; sodium lauryl sulphate; lauramine oxide; deceth-8; sodium c12-14 pareth-3 sulfate; sodium lauryl sulphate; alcohol; sodium chloride; tetrasodium glutamate diacetate; 2-propylheptanol ethoxylated; parfum; ppg-34; sodium hydroxide; phenoxyethanol; sodium bicarbonate; butylphenyl methylpropional; hexyl cinnamal; diethylenetriamine penta(methylene phosphonic acid) heptasodium salt; limonene; hydrogen peroxide; methylisothiazolinone; colorants; protease; protease stabilizer of the invention.

### Detergent Composition 3

Water; sodium lauryl sulfate; lauramine oxide; sodium laureth sulfate; ppg-26; sodium chloride; peg-8 propylheptyl ether; phenoxyethanol; styrene/acrylates copolymer; methylisothiazolinone; protease; protease stabilizer of the invention hydrogen peroxide;

### Detergent Composition 4

Water; sodium laureth sulfate; cocamidopropyl betaine; alcohol; amine; c12-18-alkyldimethyl-, n-oxide; propylene glycol; sodium chloride; sodium cumenesulfonate; parfum; kaliumacetate; sodium citrate; sorbitol; 2-bromo-2-nitropropane-1,3-diol; colorant; methylisothiazolinone; benzisothiazolinone; protease; protease stabilizer of the invention; hydrogen peroxide.

### Detergent Composition 5

Water; sodium laureth sulfate; cocamidopropyl betaine; sodium chloride; kaliumacetate; parfum; 2-bromo-2-nitropropane-1,3-diol; colorant; amylase; methylchloroisothiazolinone; methylisothiazolinone; protease; protease stabilizer of the invention; hydrogen peroxide.

1. A liquid composition comprising:
(a) a peroxide or peroxide source,
(b) a subtilisin having an amino acid sequence comprising a substitution in a position corresponding to M216 of SEQ ID NO: 1,
(c) a peptide aldehyde or ketone subtilisin stabilizer, or a hydrosulfite adduct thereof, and
(d) optionally a peroxide stabilizer.

2. The liquid composition of claim 1, wherein the amino acid sequence of the subtilisin comprises a substitution corresponding to M216A or M216S of SEQ ID NO: 1.

3. The liquid composition of claim 1, wherein the amino acid sequence of the subtilisin has at least 60% identity to the amino acid sequence of SEQ ID NO: 2.

4. The liquid composition of claim 1, wherein the peroxide concentration is in the range of 0.1% to 10%.

5. The liquid composition of claim 1, wherein the peroxide is hydrogen peroxide.

6. The liquid composition of claim 1, which is a liquid cleaning composition and further comprises a surfactant and other ingredients used in detergent and cleaning compositions.

7. The liquid composition of claim 1, which comprises a non-ionic surfactant.

8. The liquid composition of claim 1, wherein the subtilisin stabilizer has the formula P-(A)_{y}-L-(B)ₓ—B⁰—R* or a hydrosulfite adduct thereof, wherein:
a) R* is H (hydrogen), CH₃, CX₃, CHX₂, or CH₂X;
b) X is a halogen atom;
c) B⁰ is a single amino acid residue with L- or D-configuration of the formula —NH—CH(R)—C(═O)—;
d) x is 1, 2 or 3;
e) Bₓ is independently a single amino acid residue, each connected to the next B or to B⁰ via its C-terminal;
f) L is absent or independently a linker group of the formula —C(═O)—, —C(═O)—C(═O)—, —C(═S)—, —C(═S)—C(═S)— or —C(═S)—C(═O)—;
g) A is absent if L is absent or is independently a single amino acid residue connected to L via the N-terminal of the amino acid;
h) P is selected from the group consisting of hydrogen or if L is absent an N-terminal protection group;
i) y is 0, 1, or 2,
j) R is independently selected from the group consisting of C₁₋₆ alkyl, C₆₋₁₀ aryl or C₇₋₁₀ arylalkyl optionally substituted with one or more, identical or different, substituent's R′;
k) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂; and
l) R″ is a C₁₋₆ alkyl group.
m) x may be 1, 2 or 3.

9. The liquid composition of claim 8, wherein the subtilisin stabilizer is an aldehyde having the formula P—B²—B¹—B⁰—H or a hydrosulfite adduct having the formula P—B²—B¹—N(H)—CHR—CHOH—SO₃M, wherein
a) H is hydrogen;
b) B⁰ is a single amino acid residue with L- or D-configuration of the formula —NH—CH(R)—C(═O)—;
c) B¹ and B² are independently single amino acid residues;
d) R is independently selected from the group consisting of C₁₋₆ alkyl, C₆₋₁₀ aryl or C₇₋₁₀ arylalkyl optionally substituted with one or more, identical or different, substituent's R′;
e) R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂;
f) R″ is a C₁₋₆ alkyl group; and
g) P is an N-terminal protection group.

10. The liquid composition of claim 8, wherein R is such that B⁰=—NH—CH(R)—C(═O)— is Phe, Tyr or Leu; and B¹ is Ala, Gly or Val; and B² is Arg, Phe, Tyr or Trp.

11. The liquid composition of claim 8, wherein x=2, L is absent, A is absent, and P is p-methoxycarbonyl (Moc) or benzyloxycarbonyl (Cbz).

12. The liquid composition of claim 8, wherein the subtilisin stabilizer is Cbz-Arg-Ala-Tyr-H, Ac-Gly-Ala-Tyr-H, Cbz-Gly-Ala-Tyr-H, Cbz-Gly-Ala-Tyr-CF₃, Cbz-Gly-Ala-Leu-H, Cbz-Val-Ala-Leu-H, Cbz-Val-Ala-Leu-CF₃, MeO—CO-Val-Ala-Leu-CF₃, Cbz-Gly-Ala-Phe-H, Cbz-Gly-Ala-Phe-CF₃, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO₂-Phe-Gly-Ala-Leu-H, MeSO₂-Val-Ala-Leu-H, PhCH₂O—P(OH)(O)-Val-Ala-Leu-H, EtSO₂-Phe-Gly-Ala-Leu-H, PhCH₂SO₂-Val-Ala-Leu-H, PhCH₂O—P(OH)(O)-Leu-Ala-Leu-H, PhCH₂O—P(OH)(O)-Phe-Ala-Leu-H, or MeO—P(OH)(O)-Leu-Gly-Ala-Leu-H or a hydrosulfite adduct of any of these, wherein Cbz is benzyloxycarbonyl and Ac is acetyl.

13. The liquid composition of claim 8, wherein the subtilisin stabilizer is Cbz-Gly-Ala-Tyr-H or MeO—CO-Val-Ala-Leu-H, or a hydrosulfite adduct thereof, wherein Cbz is benzyloxycarbonyl.

14. The liquid composition of claim 8, wherein the peroxide stabilizer is selected from the group consisting of a carboxylic acid or carboxylate salt that comprises a short aliphatic chain of 12 carbon atoms or less or have an aromatic group, such as a phenyl group or hydroxyphenyl group; preferably, the peroxide stabilizer is chosen from acetic acid, benzoic acid, picolinic acid, salicylic acid, or sodium salicylate, and combinations thereof.

15. The liquid composition of claim 1, wherein the peroxide stabilizer is a soluble polymer selected from the group consisting of polyacrylates, polymethacrylates, polyethoxylates, polyacrylamide, polyquaterniums, and polybetaines; preferably the peroxide stabilizer a soluble cationic polymer selected from the group consisting of polyquaternium-11, polyquaternium-16, polyDADMAC, poly (acrylamido-N-propyltrimethylammonium chloride) (polyATPAC), polyoxypropylene-polyoxyethylene block copolymers, polyethylene glycols, polyacrylates, and poly(3-(3-acrylamidopropyldimethylammonio)propionate) (polyAMDAP).

16. Use of a peptide aldehyde or ketone subtilisin stabilizer, or a hydrosulfite adduct thereof, for improving the stability in a liquid hydrogen peroxide composition of a subtilisin having an amino acid sequence comprising a substitution in a position corresponding to M216 of SEQ ID NO: 1.


SEQUENCE LISTING (11 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 269 aa, Artificial
  Variant of SEQ ID NO 1
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSSATPHVAGAAALVKQKNPSWSNVQIRNHLKNTATSLGSTNLYGSGLVNAEAATR

SEQ ID NO: 3 - protein, 4 aa, Artificial
  Subtilisin inhibitor
  MISC_FEATURE (1)..(1): Acetyl-Leu
  MISC_FEATURE (4)..(4): Tyr-H
LGAY

SEQ ID NO: 4 - protein, 4 aa, Artificial
  Subtilisin inhibitor
  MISC_FEATURE (1)..(1): Acetyl-Phe
  MISC_FEATURE (4)..(4): Tyr-H
FGAY

SEQ ID NO: 5 - protein, 4 aa, Artificial
  Subtilisin inhibitor
  MISC_FEATURE (1)..(1): Acetyl-Tyr
  MISC_FEATURE (4)..(4): Tyr-H
YGAY

SEQ ID NO: 6 - protein, 4 aa, Artificial
  Subtilisin inhibitor
  MISC_FEATURE (1)..(1): Acetyl-Phe; MeO-CO-Phe; MeSO2-Phe; or EtSO2-Phe
  MISC_FEATURE (4)..(4): Leu-H
FGAL

SEQ ID NO: 7 - protein, 4 aa, Artificial
  Subtilisin inhibitor
  MISC_FEATURE (1)..(1): Acetyl-Phe or MeO-CO-Phe
  MISC_FEATURE (4)..(4): Tyr-H
FGAF

SEQ ID NO: 8 - protein, 4 aa, Artificial
  Subtilisin inhibitor
  MISC_FEATURE (1)..(1): Acetyl-Phe
  MISC_FEATURE (4)..(4): Tyr-H
FGVY

SEQ ID NO: 9 - protein, 4 aa, Artificial
  Subtilisin inhibitor
  MISC_FEATURE (1)..(1): Acetyl-Phe
  MISC_FEATURE (4)..(4): Met-H
FGAM

SEQ ID NO: 10 - protein, 4 aa, Artificial
  Subtilisin inhibitor
  MISC_FEATURE (1)..(1): Acetyl-Trp
  MISC_FEATURE (4)..(4): Tyr-H
WLVY

SEQ ID NO: 11 - protein, 4 aa, Artificial
  Subtilisin inhibitor
  MISC_FEATURE (1)..(1): MeO-P(OH)(O)-Leu
  MISC_FEATURE (4)..(4): Leu-H
LGAL